(19) United States
(12) Patent Application Publication
(10) Pub. No.: US 2024/0170160 A1
(43) Pub. Date: May 23, 2024

(54) APPLICATION OF PERSONALIZED SENSOR-BASED RISK PROFILES FOR IMPACTS OF EXTERNAL EVENTS

(71) Applicant: UnitedHealth Group Incorporated, Minnetonka, MN (US)

(72) Inventors: Gregory D. Lyng, Minneapolis, MN (US); Brian Lawrence Hill, Culver City, CA (US); James Zou, Palo Alto, CA (US); Kimmo M. Karkkainen, Santa Monica, CA (US); Kailas Vodrahalli, Los Altos, CA (US); Eran Halperin, Santa Monica, CA (US)

(21) Appl. No.: 18/211,988

(22) Filed: Jun. 20, 2023

Related U.S. Application Data
(60) Provisional application No. 63/384,923, filed on Nov. 23, 2022.

(51) Int. Cl. G16H 50/70; A61B 5/145; G16H 20/17; G16H 40/20; G16H 40/67; G16H 50/20; G16H 50/30

(52) U.S. Cl. CPC G16H 50/70; A61B 5/14532; G16H 20/17; G16H 40/20; G16H 40/67; G16H 50/20; G16H 50/30

(57) ABSTRACT

Embodiments provide for application of personalized or individualized sensor-based risk profiles for impacts of external events. An example method includes receiving sensor data from one or more sensors couplable with a subject body of a subject population comprising a plurality of subject bodies; receiving external factor data associated with the subject population; generating a population-level external event impact metric, where the population-level external event impact metric represents a predicted impact of one or more external events on a physiological or other metric of the subject population; generating a subject-level external impact metric, where the subject-level external event impact metric represents a predicted impact of the one or more external events on the physiological or other metric associated with the subject body; and initiating the performance of one or more prediction-based actions based on the subject-level external event impact metric.

### CROSS-REFERENCE TO RELATED APPLICATIONS

The present application claims priority to U.S. Provisional Application Ser. No. 63/384,923, titled “APPLICATION OF PERSONALIZED SENSOR-BASED RISK PROFILES FOR IMPACTS OF EXTERNAL EVENTS,” filed Nov. 23, 2022, the contents of which are incorporated herein by reference in their entirety.

### BACKGROUND

Diabetes mellitus is characterized by issues with the body's ability to regulate blood glucose levels. Diabetes mellitus currently impacts over 530 million people globally and can lead to numerous health complications, including cardiovascular disease, chronic kidney disease, diabetic retinopathy, and diabetic neuropathy—resulting in an annual cost of over $327 billion in the United States alone in 2017. People with diabetes are generally classified into one of two categories—Type 1 diabetes (5-10% of patients) (e.g., which is characterized by the body's inability to produce insulin), and Type 2 diabetes (90-95% of patients) (e.g., which results from a heightened resistance to insulin. People with diabetes can rely on careful diet management, medication, and/or insulin doses depending on the type of diabetes and its severity. Users use daily measurements of blood glucose to help manage glucose levels. These measurements are typically infrequent and conducted through needle pricks where a small amount of blood is used to measure the blood glucose level. However, these pointwise measurements offer a limited window into characterizing the ability to regulate glucose levels.

Through applied effort, ingenuity, and innovation, many of these identified deficiencies and problems have been solved by developing solutions that are structured in accordance with embodiments of the present disclosure, many examples of which are described in detail herein.

### BRIEF SUMMARY

Embodiments provide for application of personalized or individualized sensor-based risk profiles for impacts of external events. In some embodiments, an example method includes receiving sensor data from one or more sensors couplable with a subject body of a subject population comprising a plurality of subject bodies. The example method further includes receiving external factor data associated with the subject population. The example method further includes generating, based on applying a first trained machine learning model to the sensor data, the external factor data, and subject body vectors associated with the subject population, a population-level external event impact metric. The population-level external event impact metric represents a predicted impact of one or more external events on a physiological or other metric of the subject population. The example method further includes generating, based on applying a second trained machine learning model to the population-level external event impact metric and a subject body vector associated with the subject body, a subject-level external impact metric. The subject-level external event impact metric represents a predicted impact of the one or more external events on the physiological or other metric associated with the subject body. The example method further includes initiating the performance of one or more prediction-based actions based on the subject-level external event impact metric.

In some embodiments, one or more non-transitory computer-readable storage media are provided, including instructions that, when executed by one or more processors, cause the one or more processors to receive sensor data from one or more sensors couplable with a subject body of a subject population comprising a plurality of subject bodies, receive external factor data associated with the subject population, generate, based on applying a first trained machine learning model to the sensor data, the external factor data, and subject body vectors associated with the subject population, a population-level external event impact metric, wherein the population-level external event impact metric represents a predicted impact of one or more external events on a physiological or other metric of the subject population, generate, based on applying a second trained machine learning model to the population-level external event impact metric and a subject body vector associated with the subject body, a subject-level external event impact metric, wherein the subject-level external event impact metric represents a predicted impact of the one or more external events on the physiological or other metric associated with the subject body, and initiate the performance of one or more prediction-based actions based on the subject-level external event impact metric.

In some embodiments, a computing apparatus is provided, comprising memory and one or more processors communicatively coupled to the memory, the one or more processors configured to receive sensor data from one or more sensors couplable with a subject body of a subject population comprising a plurality of subject bodies, receive external factor data associated with the subject population, generate, based on applying a first trained machine learning model to the sensor data, the external factor data, and subject body vectors associated with the subject population, a population-level external event impact metric, wherein the population-level external event impact metric represents a predicted impact of one or more external events on a physiological or other metric of the subject population, generate, based on applying a second trained machine learning model to the population-level external event impact metric and a subject body vector associated with the subject body, a subject-level external event impact metric, wherein the subject-level external event impact metric represents a predicted impact of the one or more external events on the physiological or other metric associated with the subject body, and initiate the performance of one or more prediction-based actions based on the subject-level external event impact metric.

The above summary is provided merely for purposes of summarizing some example embodiments to provide a basic understanding of some aspects of the present disclosure. Accordingly, it will be appreciated that the above-described embodiments are merely examples and should not be construed to narrow the scope or the spirit of the present disclosure in any way. It will be appreciated that the scope of the present disclosure encompasses many potential embodiments in addition to those here summarized, some of which will be further described below. Other features, aspects, and advantages of the subject matter will become apparent from the description, the drawings, and the claims.

### BRIEF DESCRIPTION OF THE DRAWINGS

Having thus described certain example embodiments of the present disclosure in general terms above, non-limiting and non-exhaustive embodiments of the subject disclosure will now be described with reference to the accompanying drawings which are not necessarily drawn to scale. The components illustrated in the accompanying drawings may or may not be present in certain embodiments described herein. Some embodiments may include fewer (or more) components than those shown in the drawings. Some embodiments may include the components arranged in a different way:

FIG. 1 provides an example overview of a system that can be used to practice embodiments of the present disclosure.

FIG. 2 provides an example predictive data analysis computing entity in accordance with some embodiments discussed herein.

FIG. 3 provides an example external computing entity in accordance with some embodiments discussed herein.

FIG. 4 provides an example architecture that can be used to practice embodiments of the present disclosure.

FIG. 5 depicts example operations for determining and performing prediction-based actions, in accordance with some embodiments of the present disclosure.

FIG. 6 depicts example performance results associated with models predicting external factor impacts, in accordance with embodiments of the present disclosure.

FIG. 7 depicts example performance results associated with models predicting external factor impacts, in accordance with embodiments of the present disclosure.

FIG. 8 depicts an example distribution of a count of days of recorded CGM device data per patient, for use with embodiments of the present disclosure.

FIG. 9 depicts example performance results associated with models predicting external factor impacts, in accordance with embodiments of the present disclosure.

FIG. 10 depicts example performance results associated with models predicting external factor impacts, in accordance with embodiments of the present disclosure.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not necessarily indicate being based only on or based solely on the referenced element or elements unless so indicated. Like numbers refer to like elements throughout. Moreover, while certain embodiments of the present disclosure are described with reference to predictive data analysis, one of ordinary skill in the art will recognize that the disclosed concepts can be used to perform other types of data analysis.

### I. Computer Program Products, Methods, and Computing Entities

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware framework and/or operating system platform. A software component comprising assembly language instructions may require conversion into executable machine code by an assembler prior to execution by the hardware framework and/or platform. Another example programming language may be a higher-level programming language that may be portable across multiple frameworks. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as in a particular directory, folder, or library. Software components may be static (e.g., pre-established or fixed) or dynamic (e.g., created or modified at the time of execution).

A computer program product may include non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid-state drive (SSD)), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatuses, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatuses, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

### II. Example Framework

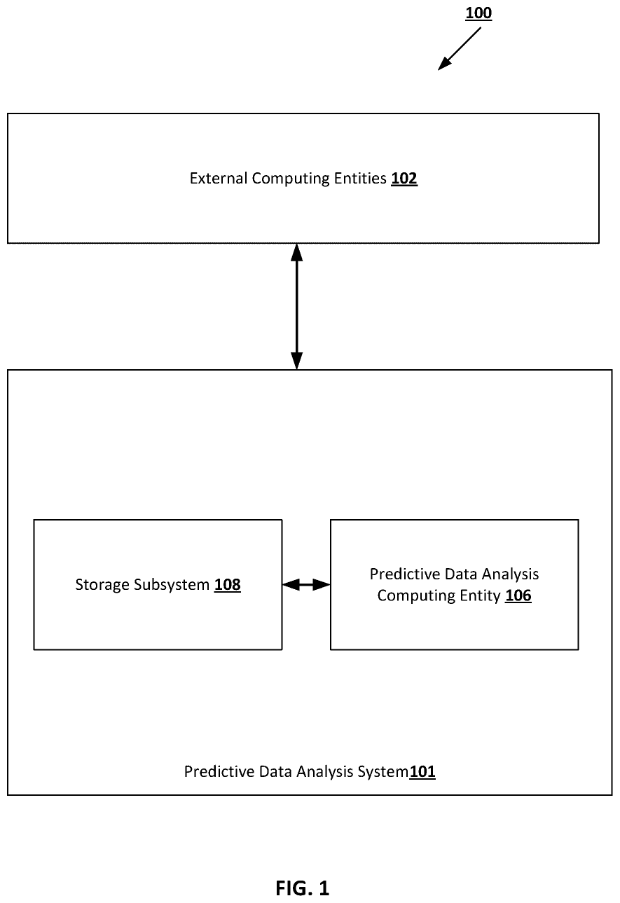

FIG. 1 provides an example overview of a system 100 that can be used to practice embodiments of the present disclosure. The system 100 may include a predictive data analysis system 101 and a predictive data analysis computing entity 106 configured to generate outputs that can be used to perform one or more output-based actions. The predictive data analysis system 101 may communicate with one or more external computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (e.g., network routers, and/or the like).

The system 100 includes a storage subsystem 108 configured to store at least a portion of the data utilized by the predictive data analysis system 101. The predictive data analysis computing entity 106 may be in communication with the external computing entities 102. The predictive data analysis computing entity 106 may be configured to: (i) train one or more machine learning models based on a training data store stored in the storage subsystem 108, (ii) store trained machine learning models as part of a model definition data store of the storage subsystem 108, (iii) utilize trained machine learning models to perform an action, and/or the like.

In one example, the predictive data analysis computing entity 106 may be configured to generate a prediction, classification, and/or any other data insight based on data provided by an external computing entity such as external computing entity 102, and/or the like.

The storage subsystem 108 may be configured to store the model definition data store and the training data store for one or more machine learning models. The predictive data analysis computing entity 106 may be configured to receive requests and/or data from at least one of the external computing entities 102, process the requests and/or data to generate outputs (e.g., predictive outputs, classification outputs, and/or the like), and provide the outputs to at least one of the external computing entities 102. In some embodiments, the external computing entity 102, for example, may periodically update/provide raw and/or processed input data to the predictive data analysis system 101. The external computing entities 102 may further generate user interface data (e.g., one or more data objects) corresponding to the outputs and may provide (e.g., transmit, send, and/or the like) the user interface data corresponding with the outputs for presentation to the external computing entity 102 (e.g., to an end-user).

The storage subsystem 108 may be configured to store at least a portion of the data utilized by the predictive data analysis computing entity 106 to perform one or more steps/operations and/or tasks described herein. The storage subsystem 108 may be configured to store at least a portion of operational data and/or operational configuration data including operational instructions and parameters utilized by the predictive data analysis computing entity 106 to perform the one or more steps/operations described herein. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

The predictive data analysis computing entity 106 can include an analysis engine and/or a training engine. The predictive analysis engine may be configured to perform one or more data analysis techniques. The training engine may be configured to train the predictive analysis engine in accordance with the training data store stored in the storage subsystem 108.

### Example Predictive Data Analysis Computing Entity

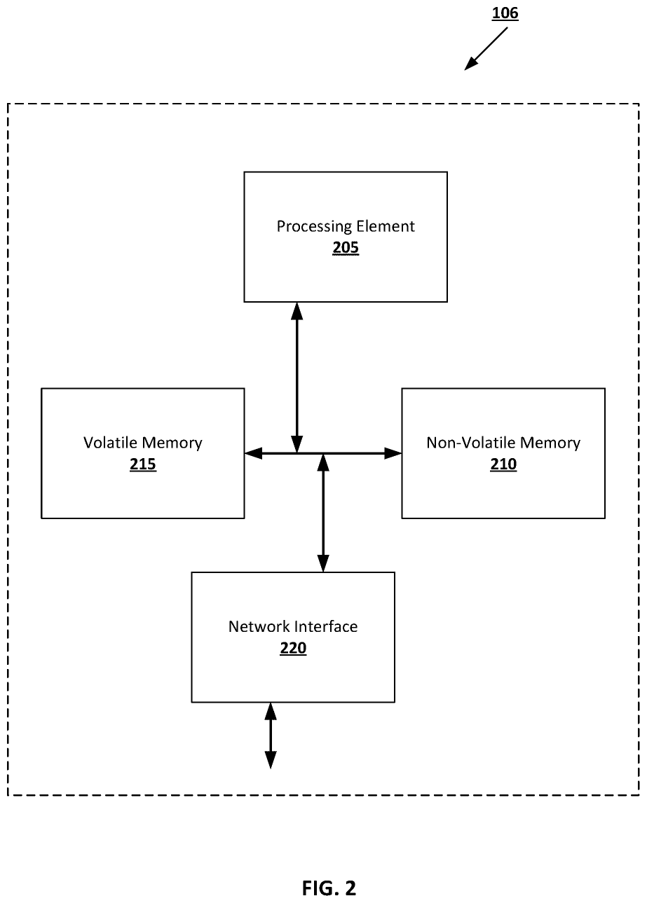

FIG. 2 provides an example predictive data analysis computing entity 106 in accordance with some embodiments discussed herein. In general, the terms computing entity, computer, entity, device, system, and/or similar words used herein interchangeably may refer to, for example, one or more computers, computing entities, desktops, mobile phones, tablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, steps/operations, and/or processes described herein. Such functions, steps/operations, and/or processes may include, for example, transmitting, receiving, operating on, processing, displaying, storing, determining, creating/generating, monitoring, evaluating, comparing, and/or similar terms used herein interchangeably. In one embodiment, these functions, steps/operations, and/or processes can be performed on data, content, information, and/or similar terms used herein interchangeably.

The predictive data analysis computing entity 106 may include a network interface 220 for communicating with various computing entities, such as by communicating data, content, information, and/or similar terms used herein interchangeably that can be transmitted, received, operated on, processed, displayed, stored, and/or the like.

In one embodiment, the predictive data analysis computing entity 106 may include or be in communication with a processing element 205 (also referred to as processors, processing circuitry, and/or similar terms used herein interchangeably) that communicate with other elements within the predictive data analysis computing entity 106 via a bus, for example. As will be understood, the processing element 205 may be embodied in a number of different ways including, for example, as at least one processor/processing apparatus, one or more processors/processing apparatuses, and/or the like.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in one or more memory elements including, for example, one or more volatile memories 215 and/or non-volatile memories 210. As such, whether configured by hardware or computer program products, or by a combination thereof, the processing element 205 may be capable of performing steps or operations according to embodiments of the present disclosure when configured accordingly. The processing element 205, for example in combination with the one or more volatile memories 215 and/or or non-volatile memories 210, may be capable of implementing one or more computer-implemented methods described herein. In some implementations, the predictive data analysis computing entity 106 can include a computing apparatus, the processing element 205 can include at least one processor of the computing apparatus, and the one or more volatile memories 215 and/or non-volatile memories 210 can include at least one memory including program code. The at least one memory and the program code can be configured to, upon execution by the at least one processor, cause the computing apparatus to perform one or more steps/operations described herein.

The non-volatile memories 210 (also referred to as non-volatile storage, memory, memory storage, memory circuitry, media, and/or similar terms used herein interchangeably) may include at least one non-volatile memory device, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile memories 210 may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity—relationship model, object model, document model, semantic model, graph model, and/or the like.

The one or more volatile memories 215 (also referred to as volatile storage, memory, memory storage, memory circuitry, media, and/or similar terms used herein interchangeably) can include at least one volatile memory device, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile memories 215 may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain embodiments of the operation of the predictive data analysis computing entity 106 with the assistance of the processing element 205.

As indicated, in one embodiment, the predictive data analysis computing entity 106 may also include the network interface 220 for communicating with various computing entities, such as by communicating data, content, information, and/or the like that can be transmitted, received, operated on, processed, displayed, stored, and/or the like. Such communication data may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless client communication networks using any of a variety of protocols, such as general packet radio service (GPRS), Universal Mobile Telecommunications System (UMTS), Code Division Multiple Access 2000 (CDMA2000), CDMA2000 1X (1xRTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

### Example External Computing Entity

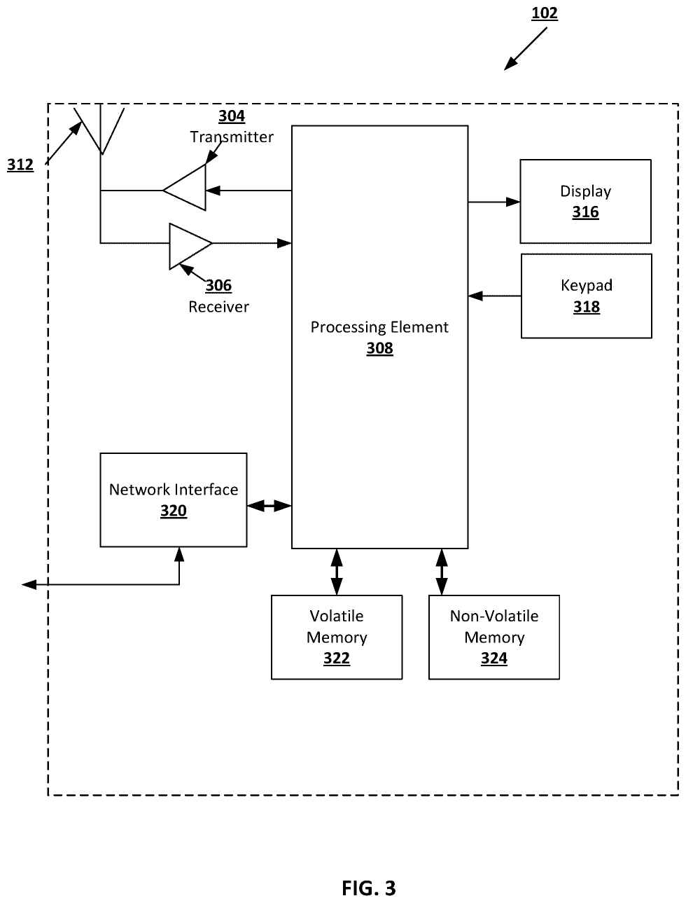

FIG. 3 provides an example external computing entity 102 in accordance with some embodiments discussed herein. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, steps/operations, and/or processes described herein. The external computing entities 102 can be operated by various parties. As shown in FIG. 3, the external computing entity 102 can include an antenna 312, a transmitter 304 (e.g., radio), a receiver 306 (e.g., radio), and/or an external entity processing element 308 (e.g., CPLDs, microprocessors, multi-core processors, coprocessing entities, ASIPs, microcontrollers, and/or controllers) that provides signals to and receives signals from the transmitter 304 and the receiver 306, correspondingly. As will be understood, the external entity processing element 308 may be embodied in a number of different ways including, for example, as at least one processor/processing apparatus, one or more processors/processing apparatuses, and/or the like as described herein with reference the processing element 205.

The signals provided to and received from the transmitter 304 and the receiver 306, correspondingly, may include signaling information/data in accordance with air interface standards of applicable wireless systems. In this regard, the external computing entity 102 may be capable of operating with one or more air interface standards, communication protocols, modulation types, and access types. More particularly, the external computing entity 102 may operate in accordance with any of a number of wireless communication standards and protocols, such as those described above with regard to the predictive data analysis computing entity 106. In a particular embodiment, the external computing entity 102 may operate in accordance with multiple wireless communication standards and protocols, such as UMTS, CDMA2000, 1xRTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the external computing entity 102 may operate in accordance with multiple wired communication standards and protocols, such as those described above with regard to the predictive data analysis computing entity 106 via an external entity network interface 320.

Via these communication standards and protocols, the external computing entity 102 can communicate with various other entities using means such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 102 can also download changes, add-ons, and updates, for instance, to its firmware, software (e.g., including executable instructions, applications, program modules), operating system, and/or the like.

According to one embodiment, the external computing entity 102 may include location determining embodiments, devices, modules, functionalities, and/or the like. For example, the external computing entity 102 may include outdoor positioning embodiments, such as a location module adapted to acquire, for example, latitude, longitude, altitude, geocode, course, direction, heading, speed, universal time (UTC), date, and/or various other information/data. In one embodiment, the location module can acquire data such as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating a position of the external computing entity 102 in connection with a variety of other systems, including cellular towers, Wi-Fi access points, and/or the like. Similarly, the external computing entity 102 may include indoor positioning embodiments, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning embodiments can be used in a variety of settings to determine the location of someone or something to within inches or centimeters.

The external computing entity 102 may include a user interface 316 (e.g., a display, speaker, and/or the like) that can be coupled to the external entity processing element 308. In addition, or alternatively, the external computing entity 102 can include a user input interface 319 (e.g., keypad, touch screen, microphone, and/or the like) coupled to the external entity processing element 308).

For example, the user interface 316 may be a user application, browser, and/or similar words used herein interchangeably executing on and/or accessible via the external computing entity 102 to interact with and/or cause the display, announcement, and/or the like of information/data to a user. The user input interface 318 can comprise any of a number of input devices or interfaces allowing the external computing entity 102 to receive data including, as examples, a keypad (hard or soft), a touch display, voice/speech interfaces, motion interfaces, and/or any other input device. In embodiments including a keypad, the keypad can include (or cause display of) the conventional numeric (0-9) and related keys (#, *, and/or the like), and other keys used for operating the external computing entity 102 and may include a full set of alphabetic keys or set of keys that may be activated to provide a full set of alphanumeric keys. In addition to providing input, the user input interface 318 can be used, for example, to activate or deactivate certain functions, such as screen savers, sleep modes, and/or the like.

The external computing entity 102 can also include one or more external entity non-volatile memories 322 and/or one or more external entity volatile memories 324, which can be embedded within and/or may be removable from the external computing entity 102. As will be understood, the external entity non-volatile memories 322 and/or the external entity volatile memories 324 may be embodied in a number of different ways including, for example, as described herein with reference the non-volatile memories 204 and/or the external volatile memories 206.

### III. Examples of Certain Terms

The term “sensor data” may refer to one or more items or streams of physiologically related data received from one or more sensors that may be coupled to or couplable to a subject body. The sensors may communicate data via client computing entities or other computing entities. A sensor may include a continuous glucose monitoring (CGM) device or other wearable sensor. Historical sensor data includes sensor data that was collected prior to a current network time.

The term “subject body” may refer to a user or subject of an analysis in accordance with embodiments herein. In some examples, a subject body may refer to a user or patient with diabetes (although a diagnosis of diabetes is not required for all embodiments or implementations). In some examples, the subject body is a human or other live subject.

The term “subject population” may refer to a group of users or subjects of an analysis in accordance with embodiments herein. In some examples, a subject population may be made of several individuals, users, or patients with diabetes (although a diagnosis of diabetes is not required for all embodiments or implementations). In some examples, the subject population contains multiple subject bodies.

The term “external factor data” may refer to one or more items of data representative of conditions associated with factors external to a subject body. For example, external factor data may include data representative of environmental conditions, weather data (e.g., precipitation, air quality index (AQI), temperature, snowfall, snow depth, humidity, and the like), and/or temporal data (e.g., holidays, birthdays, weekends, other temporal events). Historical external factor data may refer to external factor data associated with external factors that have already or previously occurred.

The term “subject body vector” may refer to a data structure comprising a plurality of records, each record containing one or more items of data associated with and/or unique to a subject body. For example, a subject vector may include clinical data, demographic data, medical history data, or other user data associated with the subject body.

The term “population-level external event impact metric” may refer to a programmatically generated quantification of a predicted impact of one or more external events on a physiological metric of a subject population. That is, using one or more trained machine learning models, a population-level external event impact metric may be generated that indicates whether and to what extent a particular external event may impact a physiological metric (e.g., glucose levels) of a subject population.

The term “physiological metric” may refer to a measure of a physiological parameter or state of a subject body. For example, a physiological metric may be a blood glucose metric. A Blood glucose metric may include time in range (TIR), interquartile range (IQR), mean amplitude of glycemic excursions (MAGE), percent coefficient of variation (% CV), or number of peaks.

The term “subject-level external impact metric” may refer to a programmatically generated quantification of a predicted impact of one or more external events on a physiological metric of a specific subject of a subject population. That is, using one or more trained machine learning models, a subject-level external event impact metric may be generated that indicates whether and to what extent a particular external event may impact a physiological metric (e.g., glucose levels) of a particular subject body.

The term “prediction-based action” may refer to automatic execution of one or more actions or workflows based on a prediction generated in accordance with embodiments herein. In some examples, a prediction-based action may include transmission of one or more alerts to computing devices, transmission of instructions to insulin delivery devices associated with the subject body, adjustments to medical equipment associated with the subject body, or adjustments to allocations of medical, computing, hospital, facility, and/or human resources to a subject body or a subject population.

The term “risk label” may refer to a classification of a subject body according to a likelihood of risk of being impacted, on a physiological level, by occurrence of a specific external event.

The term “mixed effects model” may refer to a model containing both fixed effects and random effects. In a mixed effects model, a “random slope” may refer to a metric that allows for fixed effects to vary for each subject body; a “random intercept” may refer to a metric that allows for an outcome to be higher or lower for each subject body.

The term “optimized model coefficient” may refer to the slope and/or the y-intercept of the best-fit line with respect to predicting the glycemic response metric of a user with respect to an external factor of interest. A “confidence interval” may refer to a measure of whether the given external factor of interest has a statistically significant effect at a population level.

The term “future external event” may refer to an external event that has yet to occur and is either known, certain, or predicted to occur in the future. In some examples, a future external event may include a weekend, a holiday, a change in weather conditions, a birthday, or a change in economic conditions.

The terms “trained machine learning model,” “machine learning model,” “model,” “one or more models,” or “ML” refer to a machine learning or deep learning task or mechanism. Machine learning is a method used to devise complex models and algorithms that lend themselves to prediction. A machine learning model is a computer-implemented algorithm that may learn from data with or without relying on rules-based programming. These models enable reliable, repeatable decisions and results and uncovering of hidden insights through machine-based learning from historical relationships and trends in the data. In some embodiments, the machine learning model is a clustering model, a regression model, a neural network, a random forest, a decision tree model, a classification model, or the like.

A machine learning model is initially fit or trained on a training dataset (e.g., a set of examples used to fit the parameters of the model). The model may be trained on the training dataset using supervised or unsupervised learning. The model is run with the training dataset and produces a result, which is then compared with a target, for each input vector in the training dataset. Based on the result of the comparison and the specific learning algorithm being used, the parameters of the model are adjusted. The model fitting may include both variable selection and parameter estimation. Successively, the fitted model is used to predict the responses for the observations in a second dataset called the validation dataset. The validation dataset provides an unbiased evaluation of a model fit on the training dataset while tuning the model's hyperparameters (e.g. the number of hidden units in a neural network). In some embodiments, the model can be trained and/or trained in real-time (e.g., online training) while in use.

The machine learning models as described herein may make use of multiple ML engines, e.g., for analysis, transformation, and other needs. The system may train different ML models for different needs and different ML-based engines. The system may generate new models (based on the gathered training data) and may evaluate their performance against the existing models. Training data may include any of the gathered information, as well as information on actions performed based on the various recommendations.

The ML models may be any suitable model for the task or activity implemented by each ML-based engine. Machine learning models may be some form of neural network. The underlying ML models may be learning models (supervised or unsupervised). As examples, such algorithms may be prediction (e.g., linear regression) algorithms, classification (e.g., decision trees, k-nearest neighbors) algorithms, time-series forecasting (e.g., regression-based) algorithms, association algorithms, clustering algorithms (e.g., K-means clustering, Gaussian mixture models, DBscan), or Bayesian methods (e.g., Naïve Bayes, Bayesian model averaging, Bayesian adaptive trials), image to image models (e.g., FCN, PSPNet, U-Net) sequence to sequence models (e.g., RNNs, LSTMs, BERT, Autoencoders) or Generative models (e.g., GANs).

Alternatively, ML models may implement statistical algorithms, such as dimensionality reduction, hypothesis testing, one-way analysis of variance (ANOVA) testing, principal component analysis, conjoint analysis, neural networks, support vector machines, decision trees (including random forest methods), ensemble methods, and other techniques. Other ML models may be generative models (such as Generative Adversarial Networks or auto-encoders).

In various embodiments, the ML models may undergo a training or learning phase before they are released into a production or runtime phase or may begin operation with models from existing systems or models. During a training or learning phase, the ML models may be tuned to focus on specific variables, to reduce error margins, or to otherwise optimize their performance. The ML models may initially receive input from a wide variety of data, such as the gathered data described herein.

The terms “data,” “content,” “digital content,” “digital content object,” “signal,” “information,” and similar terms may be used interchangeably to refer to data capable of being transmitted, received, and/or stored in accordance with embodiments of the present disclosure. Thus, use of any such terms should not be taken to limit the spirit and scope of embodiments of the present disclosure. Further, where a computing device is described herein to receive data from another computing device, it will be appreciated that the data may be received directly from another computing device or may be received indirectly via one or more intermediary computing devices, such as, for example, one or more servers, relays, routers, network access points, base stations, hosts, and/or the like, sometimes referred to herein as a “network.” Similarly, where a computing device is described herein to send data to another computing device, it will be appreciated that the data may be transmitted directly to another computing device or may be transmitted indirectly via one or more intermediary computing devices, such as, for example, one or more servers, relays, routers, network access points, base stations, hosts, and/or the like

### IV. Overview, Technical Improvements, and Technical Advantages

Embodiments herein provide for demonstration of population-level significance for various external factors in inhibiting or supporting glucose management. Embodiments herein further provide for predicting individual user likelihood of being at risk of glucose-related events (e.g., reduction in time in range (TIR)) from external factors. Based on the programmatically generated likelihood, one or more prediction-based actions can be initiated so that the appropriate action is taken at the appropriate time for the individual user, thereby avoiding or mitigating the glucose-related event or events.

Continuous glucose monitor (CGM) devices generate periodic and/or frequent measurements (e.g., every five minutes) of interstitial glucose levels in an individual's (e.g., also referred to herein as a user or patient) body. GCM devices may support self-management and behavior change by providing individuals visibility into whether and to what extent various internal and/or external factors (e.g., meals, exercise, activity, sleep, stress, and medication adherence) impact their own glucose levels. That is, over time, engaged individuals may learn the difference between the glycemic impact (e.g., specific to the individual) of, for example, consuming a bowl of sugary cereal and that of taking a postprandial walk.

Individuals having Type 2 diabetes (T2D) are advised that they should aim for 70% of their sensor readings of blood glucose levels to fall between 70 and 180 mg/dL. A corresponding measure, time in range (TIR) (representing the time spent in the target range between 70 and 180 mg/dL) is a useful metric in CGM-based T2D disease management because TIR provides a standardized metric for diabetes patients. However, TIR results from a complex interplay of a variety of factors at both the physiological and environmental levels. Healthy eating, exercise amount and frequency, sleep quality, stress levels, and medication adherence can impact glycemia and resulting CGM-based metrics of interest. Repeated measurements of daily TIR may display within-subject variability due to changes in daily activities and diet (in addition to normal biological fluctuations). These human behaviors may naturally change when circumstances change (e.g., weekday vs. weekend, holidays, good weather, bad weather, etc.). In addition, individuals with diabetes have been reported to have lower skin blood flow and sweating responses during heat exposure, and it has been suggested that high temperatures have a physiological impact on glycemic control in diabetics.

While TIR may provide a standardized metric, it does not provide a granular metric capable of providing insight into times and reasons for variations in glucose levels. Without granular insight into times and reasons for variations in glucose levels, efficient and appropriate provisioning of resources (e.g., computing, automated equipment, or otherwise) associated with avoiding or mitigating glucose events is nearly impossible and is fraught with mistakes (e.g., overly conservative resulting in wasted resources; overly aggressive resulting in mismanagement of resources).

Embodiments herein provide for the identification—based on CGM device readings and demographic variables—of individuals whose TIR (e.g., or other CGM-derived metric of interest) is likely to be negatively impacted by external events. For example, individuals who effectively control their glucose levels during normal daily and weekly routines may behave differently during semi-rare events such as holidays, weekends, unexpected weather events, or changes in environmental conditions.

Embodiments further provide for allocation of resources in a manner most efficient to individuals based on, for example, individuals most likely to see a negative impact on TIR due to upcoming or existing external factors. That is, a likelihood that an individual will experience a negative impact on their TIR as a result of upcoming or predicted external factors can be programmatically generated using models configured in accordance with embodiments herein. Subsequently, based on the programmatically generated likelihood, one or more prediction-based actions can be initiated so that the appropriate action is taken at the appropriate time for the individual (e.g., as opposed to according to approximations). Thus, embodiments of the present disclosure reduce wasted resources and avoid possible catastrophes associated with diabetes management.

### V. Example System Operations

Embodiments of the present disclosure relate to determining which individuals are most impacted by various external factors (e.g., weather conditions, temporal events), quantifying the impact of the external factors, and initiating performance of prediction-based actions based on whether, and to what extent, an individual is, or individuals are or will be impacted.

Embodiments herein determine how users at a population level are or will be impacted, in terms of glucose levels, by existing or predicted external factors (e.g., temperature, holidays, weekends). Not only do embodiments herein provide evidence that external factors can impact blood glucose levels, but embodiments herein also provide evidence demonstrating how user behavior may be characterized by a user's environment and how such environment can impact the user's ability to manage glucose.

Embodiments herein apply one or more trained machine learning models to subject body (e.g., user) vectors (e.g., comprising records representative of data associated with a user's medical history, CGM device data, and/or other wearable device data collected from sensor devices coupled or couplable with a user's body) to determine individual users whose glycemic metrics are likely to be significantly impacted by various external factors including environmental (e.g., temperature, rain, snow, precipitation, humidity, pressure, wind, extreme weather events like heat waves or ice freezes), temporal (e.g., holidays, weekends, other dates of interest to a particular individual, or other behaviorally driven temporal dates/events), or other events or factors. The predictions provide for early warnings of risks and provide for efficient allocation of resources or execution of prediction-based actions for mitigating or avoiding significantly impacted glucose levels specific to individuals.

It will be appreciated that, while embodiments herein are described with respect to CGM device data and/or other wearable device data, models herein may be trained, retrained, and/or applied to one or more additional other types of sensor or other data outside of those listed herein without departing from the scope of the present disclosure. Further, clinical and demographic data may be supplemented or replaced by other intermittent and static information about an entity that is not necessarily an individual. An external event may be any event of interest (e.g., a sudden change in stock prices).

### Example Datasets and Metrics

In some embodiments, extensive datasets including CGM data paired with wearables data (e.g., heart rate and step count) and medical records (e.g., demographic information, disease history, medications, and claims data) are used for each user or individual. Embodiments further leverage publicly available datasets containing historical daily weather data and historical daily air quality data. The datasets are pre-processed and joined for use in implementations herein. The datasets described herein may be used to train and/or re-train models described herein, and the datasets described herein may be inputs to the models described herein.

### CGM and Activity Tracker Data

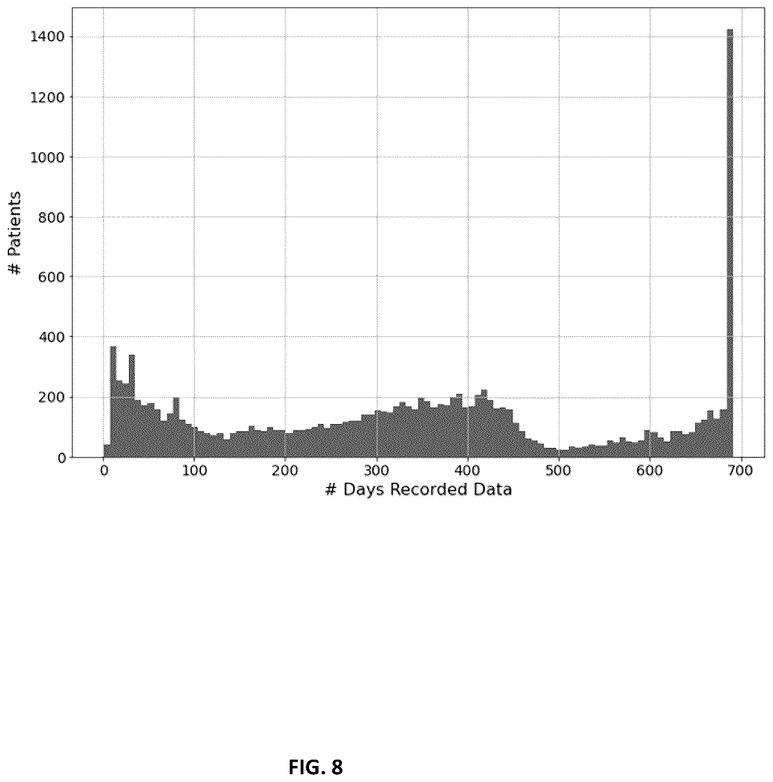

In some embodiments, glucose metrics are tracked through a CGM device for a given duration according to a period (e.g., at least every 10 days every 3 months). Examples of general demographic information are provided in TABLE 1 an example distribution of number of days of data recorded per user is depicted in FIG. 8. That is, FIG. 8 depicts an example distribution of a count of days of recorded CGM device data per user, for use with embodiments of the present disclosure.

In some embodiments, the CGM devices are configured to measure blood glucose level periodically (e.g., once every 5 minutes over the duration the device is worn).

In some embodiments, preprocessing of the CGM device data includes partitioning the dataset into individual days in implementations where analyses are conducted relative to a 24-hour window of data (midnight to midnight). For example, a single day may be selected, in some embodiments, as there may be a clear glucose cycle within a single day period. In each given partitioned period, recorded data may be interpolated at smaller (e.g., 5-minute intervals), starting from a given starting timestamp (e.g., midnight). For a single day, with 5-minute intervals, 288 datapoints per day per user are available. Partitions with a threshold amount of missing data points may be excluded from the dataset. Preprocessing of activity tracker (e.g., or other sensor) data may be partitioned according to the description above. In some embodiments, activity tracker and/or other sensor data is not excluded. Each user also has associated medical history, medication, and claims data. This data may be preprocessed to be in an ingestible format for embodiments herein.

In some embodiments, each user is associated with at least one geographic code (e.g., zip code or postal code) based on a location of medical care received. In some examples, users associated with geographic codes that differ in location by more than a certain radius (e.g., 10 miles) during a duration of time associated with the dataset (e.g., a 2-year period used in the analysis) may be excluded. This exclusion may done to facilitate joining with environmental data that is linked by geographic code.

### Weather and Air Quality Data

For ease in description and brevity, an example weather dataset and air quality dataset are described herein together as they may be relatively similar datasets, and may be similarly preprocessed.

In some embodiments, weather measurements include maximum temperature (TMAX), minimum temperature (TMIN), amount of rainfall (PRCP), amount of snowfall (SNOW), and depth of snow (SNWD). It will be appreciated that SNOW refers to snowfall on a given day, while SNWD refers to how much snow is on the ground on the given day (e.g., which may include a previous day's snowfall). Air quality data may include a measurement for the air quality index (AQI), which is calculated based on the concentration of small particulates. Examples of general statistics for these datasets is shown in TABLE 2.

In some embodiments, weather measurements is recorded at base stations or other locations periodically (e.g., daily). Base stations may have fixed, known locations (e.g., latitude and longitude are known). However, each base station may not provide a measurement on each day or for each period.

In some embodiments, to join environmental condition datasets with user measurement datasets, the closest available weather measurement to a user's geographic code on each day the user has sufficient CGM measurements is used. Because base stations may not provide measurements every day, the base stations used may vary across a user's history.

### Extreme Weather and Air Quality Data

In addition to the continuous weather measurements, it may be beneficial to glean insights into user and/or glucose level behavior relative to extreme weather events (e.g., heatwaves). A heatwave is considered to include anomalous high temperatures relative to location and time of year. This may be used as a standard to calculate extreme weather events for each of the six weather conditions.

In some embodiments, for each weather condition (e.g., those described above), regional clusters are generated or defined. These clusters may be based on geographically clustering a population of users into multiple (e.g., 20) groups and defining the clusters as center points for regions. Within each cluster, an extreme weather event may then be defined as outside two standard deviations of the mean over the period of the collected data. To define an extreme weather event, in some examples, at least three consecutive days of extreme weather within a 10-mile radius may be desirable. Controls may be employed in the analysis.

### Temporal Events

In some embodiments, examples of temporal events used in example data analyses herein include events associated with certain dates during a year (e.g., holidays and weekends). Non-limiting examples of holiday events include July 4th, Halloween (October 31st), Thanksgiving (fourth Thursday in November), New Year, and the user's birthday. An entire week of days for each holiday may be included, allowing for “holiday-like” behavior over multiple days (e.g., eating Thanksgiving leftovers later in the week). Controls may be employed in the analysis.

### Glucose Metrics

In some embodiments, various metrics for quantifying blood glucose management are considered. These include time in range (TIR), inter quartile range (IQR), mean amplitude of glycemic excursions (MAGE), percent coefficient of variation (% CV). Definitions of each are provided below. Here gₜ refers to a user's glucose measurement (in mg/dL) at time t on a given day; μ_{g}, σ_{g} refer to the mean and standard deviation of gₜ on a given day; and Q₁, Q₃ refer to the 25^{th} and 75^{th} percentiles respectively.

$$TIR = 100 \% \cdot E [ 1 [ 70 \frac{mg}{dL} \leq {g}_{t} \leq 180 \frac{mg}{dL} ] ] \qquad ( 1 )$$

$$IQR = {Q}_{3} ( {g}_{t} ) - {Q}_{1} ( {g}_{t} ) \qquad ( 2 )$$

$$MAGE = E [ \vert {g}_{t} - {μ}_{g} \vert \cdot 1 [ \vert {g}_{t} - {μ}_{g} \vert > {σ}_{g} ] ] \qquad ( 3 )$$

$$\% CV = 100 \% \cdot \frac{{σ}_{g}}{{μ}_{g}} \qquad ( 4 )$$

In some embodiments, additional metrics are employed including the mean, standard deviation, 90^{th} percentile (a slightly more robust version of the maximum value), and a count of glucose peaks. Measurements may be calculated with respect to a single day (e.g., or other chosen duration) for a single user. TABLE 2 includes example ranges of values observed in an example dataset, desired ranges of values for the average user, and whether an increase or decrease is generally considered healthy.

### Population-Level Metrics

To understand whether any of the external factors have a statistically significant effect at a population-level effect, a linear mixed effects model can be employed. An example model may assume:

CGM_{metric}β₀+βₑxₑₓₜₑᵣₙₐₗ+ [symbol] \vec{β}_{d},\vec{x}_{d} [symbol] +α_{0,i}+α_{e,i}xₑₓₜₑᵣₙₐₗ+ϵ (5)

where CGM_{metric} is the metric of interest and α, β are the fitted random and fixed effect parameters respectively. In particular, βs are the fixed effects with β₀, βₑ, \vec{β}_{d} being the population intercept, effect due to the external factor, and effect due to demographic information. Similarly, α_{0,i} and α_{e,i} are the random intercept and slope specific for user i. Together, these terms can be thought of as a user-specific adjustment to better model user i. Finally, ϵ is a Gaussian noise term.

A mixed effects model corrects for the fact that the data is not independent. Recall that each datapoint is a single day of data from a single user. As there are multiple days of data coming from each user, there will be dependence between data coming from the same user.

In some embodiments, a separate model for each external factor and glucose metric pair under consideration is fit. This results in 168 separate βₑ models. External factors which have a significant population-level effect are determined by identifying when the null hypothesis that the effect is zero can be rejected (p-value<0.05). Here, the p-value is used which is based on a z-test where mean and βₑ are estimated as part of fitting the mixed effects model. Multiple hypothesis testing using the Bonferroni correction is corrected for.

### User-Level Metrics

In some embodiments, given the aforementioned population-level models, a user-level classifier is constructed to identify which users are most significantly impacted by each external factor. Note that such users can be identified regardless of whether the external factor has a significant population-level effect (e.g., users are highly heterogenous, and some of that heterogeneity can be identified herein).

In some embodiments, for each external factor and glucose metric pair, users are separated into multiple groups (e.g., three groups—(i) those who have a stronger glycemic response, (ii) those who have a weaker glycemic response, and (iii) those who have a similar response to the external factor relative to a population average). This separation or segmentation may be based on random slopes for the corresponding mixed effects model (e.g., random slopes are user-level corrections to the population-level effect due to the external factor). The population of random slopes may also be partitioned into multiple groups (e.g., three groups—(a) those at least one standard deviation below the mean, (b) above the mean, or (c) within one standard deviation of the mean).

In some embodiments, a label is then assigned to users based on their partition and a random forest classifier is trained to predict this label given basic demographic information and features derived from the CGM and wearable devices. The label is used to determine whether an individual has worse glycemic management with respect to an external factor relative to what is typical for the population. That is, the three groups are converted into a binary label—those that have worse glycemic management (e.g., at least one standard deviation below the mean) and those that do not (e.g., everyone else). To report results, the receiver operating characteristic area under curve (ROC-AUC) and precision recall area under curve (PR-AUC) metrics are used. As the classifiers each predict a binary label, ROC-AUC and PR-AUC values are reported using the standard computation for binary classifiers.

### Example Operations and Architecture

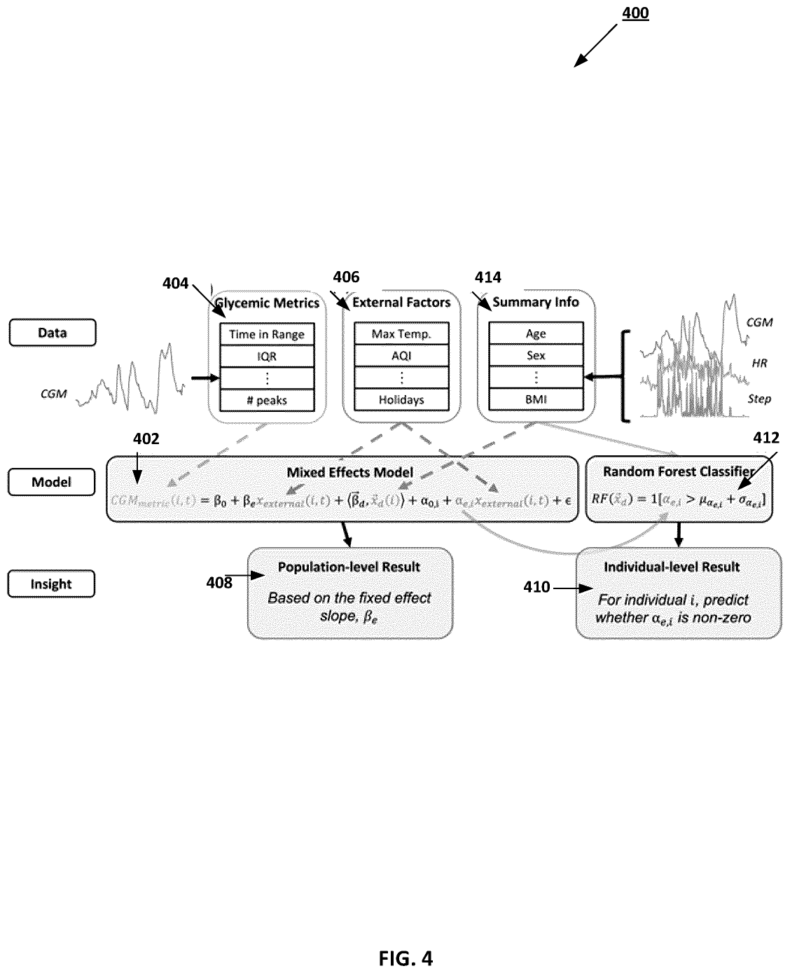

FIG. 4 depicts an example architecture 400 for use with embodiments of the present disclosure. In some embodiments, example architecture 400 is implemented in accordance with a predictive data analysis computing entity 106.

In FIG. 4, an example architecture 400 may include a mixed effects model 402, configured to receive inputs including sensor data such as physiological sensor data 404 (e.g., glucose metrics such as time in range, IQR, a number of glucose level peaks, and the like) as well as external factor sensor data 406 (e.g., temperature, maximum temperature, AQI, temporal event data such as holidays and the like). The mixed effects model 402 may be configured to determine, based on the inputs (e.g., 404, 406), a population-level result 408 as well as a user level result 410. The user level result 410 may be further based on a trained random forest classifier 412 (e.g., or other machine learning model) as well as additional inputs including, for example, user features 414.

In some embodiments, the sensor or user specific inputs (e.g., 404) to the mixed effects model 402 include clinical data (e.g., ICD history and prior medication information), demographic information (e.g., age, sex, gender, geographic location, BMI, A1C, height, weight), and/or digital signals (e.g., CGM device or other wearable recordings and movement and heart rate data) as described herein. The external factor data inputs (e.g., 406) may include historical data and sometimes data specific to the region within which the user resides or spends significant time. The inputs (e.g., 404, 406) may be used for predicting population level effects 408 and/or segmenting the population in those who are or may be most impacted by the external factors.

In some embodiments, for one or more external factors of interest (e.g., a holiday or weather event) 406, coefficients of the model 402 are optimized to configure the model 402 to predict a measure of glycemic control (e.g., fraction of time in range) 408 for a population (e.g., or subset of a population). In some embodiments, the population-level result 408 is based in part on a fixed effect slope βₑ.

In some embodiments, inputs to the model 402 include a subset of the inputs described above (e.g., 404) as well as a single external factor of interest (e.g., a subset of 406) (e.g., the daily maximum temperature). With respect to the subset of inputs (e.g., subset of 404), in some embodiments, each datapoint for the model 402 may include: (a) a limited amount of demographic data related to the user; (b) a single external event of interest on a given day; and (c) one glycemic response metric computed on the same given day for the user. In some embodiments, reducing the inputs to the aforementioned datapoints may reduce computing power and increase efficiency of the model. However, the model need not be limited to a subset of input data. While embodiments herein are described and/or depicted with respect to an example linear mixed effects model, other potentially non-linear hierarchical models can be used without departing from the scope of the present disclosure

In some embodiments, one or more models 402 use random intercepts and random slopes (e.g., for the external factor) per individual to control for repeated measures. As used herein, random intercepts and random slopes may refer to quantities that measure individual deviations from population-level predictions. That is, random slopes may be associated with an individual and reflect individual level deviation from the population response. The outputs may include optimized model coefficients along with confidence intervals for each of the coefficients.

In some embodiments, optimized model coefficients include (a) the slope and (b) the y-intercept of the best-fit line with respect to predicting the glycemic response metric of the user with respect to the external factor of interest. The confidence intervals may provide for determining those external factors of interest having a significant impact an individual's glycemic response metric. That is, a confidence interval may indicate whether the given external factor of interest (e.g., an individual's birthday) has a statistically significant effect at a population level.

In some embodiments, subsets of individuals who are most impacted by each external factor are identified. In some examples, the random slopes may be used to segment a population into multiple groups (e.g., three groups—(i) those who have a stronger glycemic response, (ii) those who have a weaker glycemic response, and (iii) those who have a similar response to the external factor relative to a population average). A random forest classifier 412 may then be trained, given user features 414, to predict to which group an individual belongs given the full set of inputs (e.g., 404). For predicting the group to which the individual belongs, in some embodiments, multiple (e.g., 3-10) days' worth of digital signal data (e.g., CGM data) may be used. In embodiments, this can be generated regardless of whether the external factor had a significant effect at the population level and/or whether the external factor was observed (e.g., in the case of an event such as a holiday) in the recorded digital signal data for the individual. Indeed, various embodiments of the present disclosure may identify segments of the population for which the external factor does have a significant effect while there is an absence of a significant effect at the population level. In some embodiments, the user-level result 410 is based on part on a prediction as to whether α_{e,i} is non-zero.

It will be appreciated that, while embodiments herein reference and describe a random forest classifier implementation, other models or techniques (e.g., linear models, deep neural networks, elastic net regression, clustering, k-means classification, support vector machines, latent Dirichlet allocation, and/or the like) may be used without departing from the scope of the present disclosure.

In some embodiments, the trained random forest classifier 412 (e.g., or other classifier) is applied to a user feature vector 414 associated with an individual to predict which external factors are likely to have a significant impact on the individual's glycemic control, producing a user-level result 410. For the feature vector associated with the individual, embodiments of the present disclosure may include, among other digital signal data, inputs 404.

In some embodiments, the trained random forest classifier 412 (e.g., or other classifier) is incorporated into an early warning system to allow for automatic flagging of individuals at risk for various external factors and for initiation of one or more prediction-based actions based on the predictions from the classifier 412. Examples of prediction-based actions may include transmission of alerts to computing devices associated with individuals and/or health coaches or clinicians who advise the individuals to allow early interventions (e.g., a low daily temperature is predicted in a weather forecast, and using knowledge about an individual, they are flagged of being at risk for lower glycemic control on a given day), transmission of instructions to insulin delivery devices associated with an individual, automatic adjustments to medical equipment associated with the individual, adjustments to allocations of medical, computing, hospital, facility, and/or human resources to an individual, population, subset of a population, and/or the like.

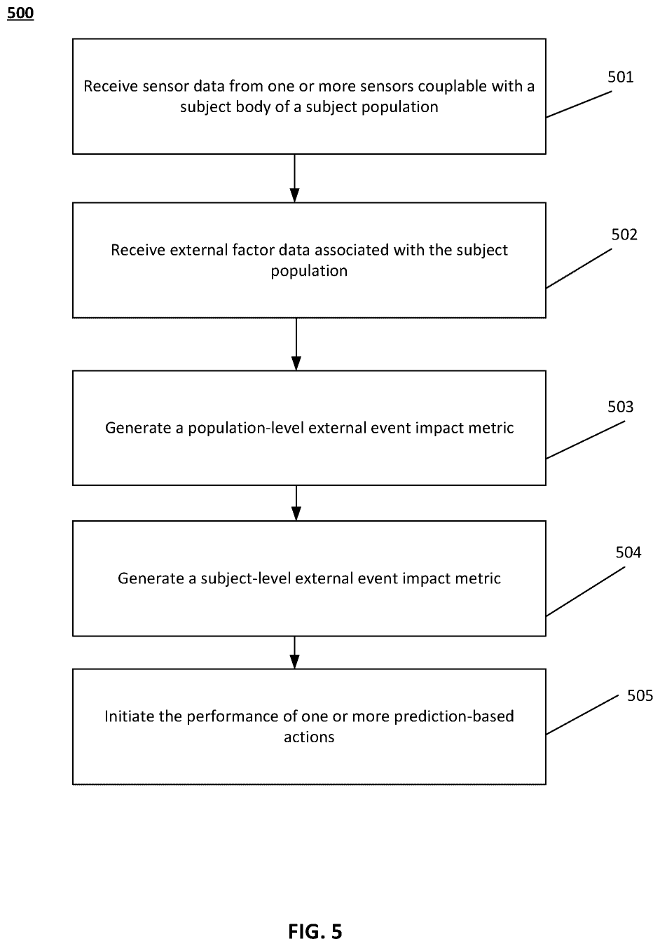

FIG. 5 depicts example operations 500 for determining and performing prediction-based actions, in accordance with some embodiments of the present disclosure. Via the various steps/operations of the process 500, a predictive analysis computing entity 106 can efficiently and effectively make real-time decisions for initiating the performance of prediction-based actions aimed at mitigating or avoiding possibly detrimental glucose-related events. The initiation of the performance of prediction-based actions can be caused based on a prediction as to whether and to what extent a population or a specific user of a population will experience a glucose-level impact as a result of one or more external events occurring. The prediction-based actions can be initiated based on predictions in advance of the external events, thereby providing opportunity to mitigate or avoid the glucose-related event.

In some embodiments, the process 500 begins at step/operation 501 when the predictive data analysis computing entity 106 receives sensor data. For example, the predictive analysis computing entity 106 may receive the sensor data from one or more sensors couplable with one or more subject bodies of a subject population. In some examples, the sensors may include a continuous glucose monitoring (CGM) device) and the sensor data may be collected and transmitted to the predictive analysis computing entity 106 according to a period (e.g., every 5 minutes) for a given duration (e.g., 3 days).

In some embodiments, the process 500 continues at step/operation 502 when the predictive data analysis computing entity 106 receives external factor data associated with the subject population. For example, the predictive data analysis computing entity 106 may receive weather measurements from base stations, weather sensors, environmental sensors, or other locations periodically (e.g. daily or hourly). The predictive data analysis computing entity 106 may further receive location data as well as temporal event data from a client computing entity associated with a user and/or from a repository. It will be appreciated that, while embodiments herein provide examples of temporal events (e.g., holidays, birthdays, weekdays, weekends), temporal events may be configurable in various embodiments (e.g., users may designate specific temporal events according to preferences).

In some embodiments, the process 500 continues at step/operation 503 when the predictive data analysis computing entity 106 generates a population-level external event impact metric. For example, the predictive data analysis computing entity 106 may generate a population-level external event impact metric by applying a trained machine learning model (e.g., a linear mixed effects model) to the received sensor data, the external factor data, and subject body vectors (e.g., including demographic, clinical, medical, and other data associated with subject bodies) associated with the subject population. The population-level external event impact metric represents a predicted impact of one or more external events on a physiological metric of the subject population. In some examples, a different trained machine learning model is used for each external factor of the one or more external factors.

In some embodiments, the process 500 continues at step/operation 504 when the predictive data analysis computing entity 106 generates a subject-level external event impact metric. For example, the predictive data analysis computing entity 106 may generate the subject-level external event impact metric by applying a second trained machine learning model (e.g., a random forest classifier) to the population-level external event impact metric and a subject body vector associated with a specific subject body. The subject-level external event impact metric represents a predicted impact of the one or more external events on the physiological metric associated with the specific subject body.

In some embodiments, the process 500 continues at step/operation 505 when the predictive data analysis computing entity 106 initiates the performance of one or more prediction-based actions based on the subject-level external event impact metric. In some examples, the predictive data analysis computing entity 106 may initiate the performance of one or more prediction-based actions based on the population-level external event impact metric. Such prediction-based actions may include automated alerts, automated instructions to insulin delivery devices, automated adjustments to medical equipment, automated adjustments to allocations of medical, computing, hospital, facility, and/or human resources. Further, such automated actions may include automated physician notification actions, automated patient notification actions, automated appointment scheduling actions, automated prescription recommendation actions, automated drug prescription generation actions, automated implementation of precautionary actions, automated record updating actions, automated datastore updating actions, automated hospital preparation actions, automated workforce management operational management actions, automated server load balancing actions, automated resource allocation actions, automated call center preparation actions, automated hospital preparation actions, automated pricing actions, automated plan update actions, automated alert generation actions, and/or the like.

### Example Results

A large dataset of CGM measurements collected from a cohort of adults with Type 2 diabetes over a particular time period (e.g., 2-year period) was utilized. In total, over 10,000 users and over 1,000,000 days of CGM data were collected. This data was paired with health records including medical claims data, as well as wearable data (including heart rate information and step counts). This data was paired with external datasets to understand the environmental conditions a user experienced on any given day. The resulting dataset was then analyzed to understand how a users' ability to manage glucose is impacted by external factors including weather conditions and temporal events like holidays or weekends.

### Population-Level Results

For the population-level results, all external factors were selected that have a significant effect (p-value<0.05) on TIR. TABLE 3 shows the results for these selected factors from the models with respect to all glucose metrics for those factors. There are three external factors that are associated with a significant effect at a population level: maximum temperature (e.g., higher temperature leads to better glucose management; higher temperature has a physiological effect that can increase glucose absorption by the body), holidays (e.g., holidays harm a user's ability to manage glucose) and weekends (e.g., users are worse at managing glucose on weekends). While each metric offers only a one-dimensional view into the complexity of a user's glucose signal, the fact that the analysis shows significant change related to the external events further suggest the effect of the external event is significant and has a measurable impact on user health.

### User-Level Results

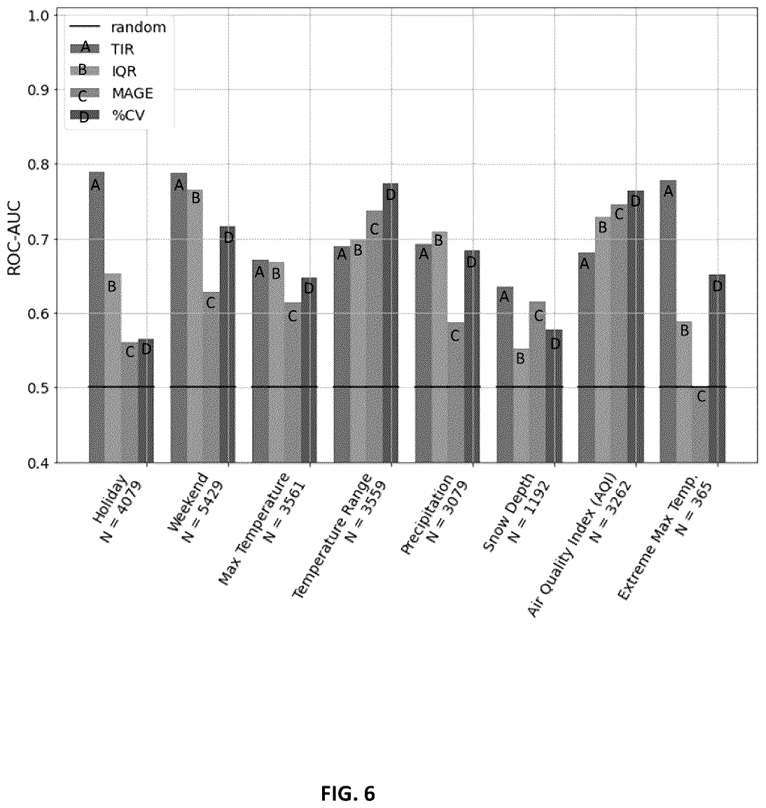

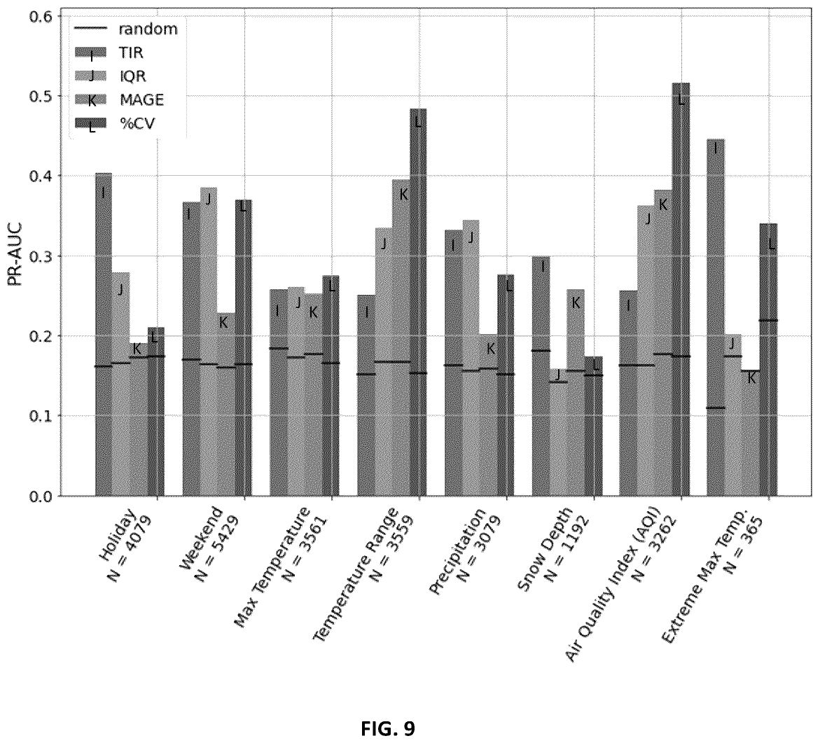

FIG. 6 depicts example performance results associated with models predicting external factor impacts, in accordance with embodiments of the present disclosure, with respect to twelve different event types (each listed on the horizontal axis). The event types include both temporal events (e.g., holidays and weekends) and environmental ones (e.g., precipitation, snow depth, air quality index (AQI), extreme maximum temperature, maximum temperature, and temperature range). AQI stands for Air Quality Index and denotes events related to air pollution. The performance metric for each is the area under the receiver-operator curve (ROC-AUC) (see FIG. 9 for the same plot with PR-AUC). Performance is measured with respect to five measurements of glucose control Time in Range (TIR), Interquartile Range (IQR), Mean Amplitude of Glycemic Excursions (MAGE), and percent coefficient of variation (% CV). FIG. 6 illustrates that embodiments herein perform better than random (represented by the solid horizontal line) for most event types.

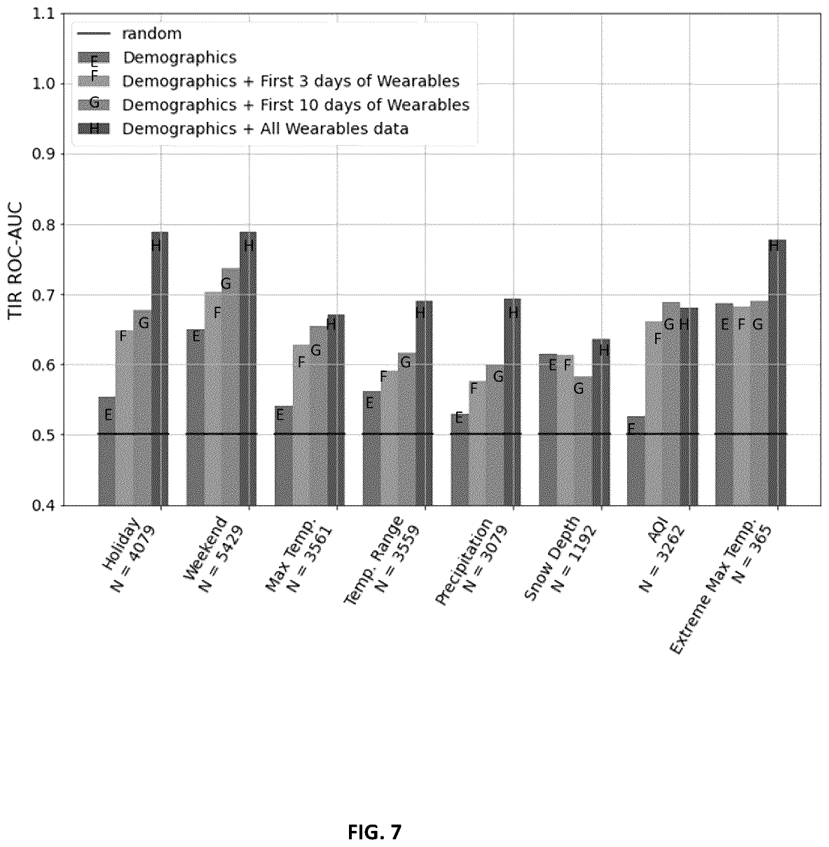

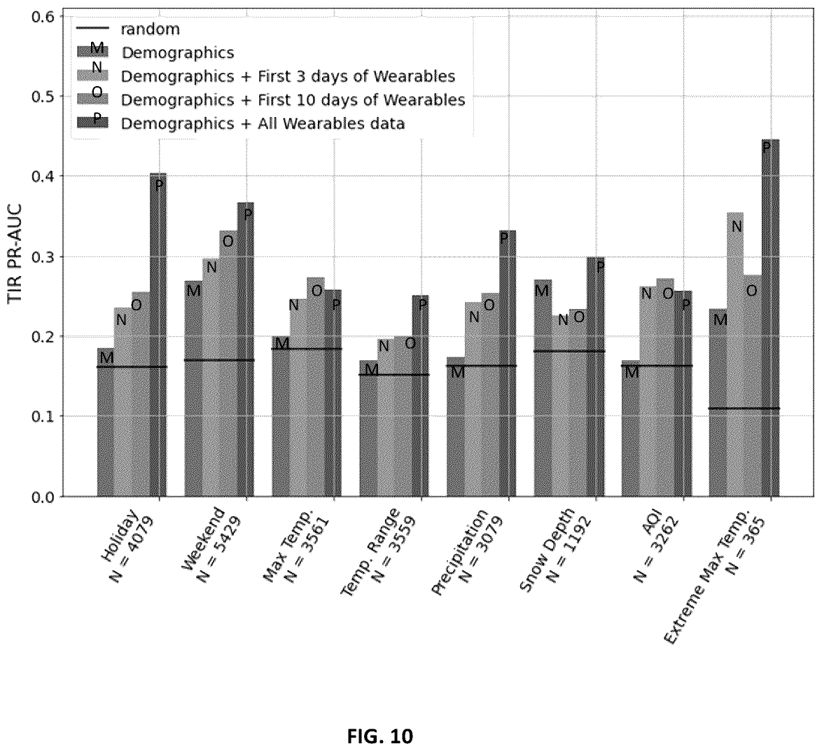

FIG. 7 depicts example performance results associated with models predicting external factor impacts, in accordance with embodiments of the present disclosure. That is, the extent to which availability of CGM and wearables data can improve prediction performance (ROC-AUC) is compared in FIG. 7 (see FIG. 10 for the same plot with PR-AUC). The models are plotted using one of four feature sets: only demographic features (E), demographic features and summary statistics based on three (F), ten (G), or all days (H) of CGM and wearables data. Note that performance consistently improves given more CGM and wearables data, and that performance is already high given only ten days of data. Having access to all wearables data, even though all days are aggregated into a single set of summary values, consistently improved performance. It is important to note, however, that even with just 3 days of data, close to 0.70 AUC was observed for more than half of the external factors. TABLE 4 summarizes Summary of external events and the range of values that occurred in the data. The results herein underscore the ability of embodiments herein to predict user response to external factors relative to their ability to regulate glucose.

TABLE 1
Demographic information of Type 2 Diabetes user dataset after filtering.

| | | # Users | # Days | Age (μ ± σ) | Sex (% M/% F) |
|---|---|---|---|---|---|
| Total | | 5447 | 940663 | 55.58 (+/−8.78) | 48.84%/51.16% |
| Weather | Temp. Max | 3561 | 506260 | 55.72 (+/−8.79) | 51.92%/48.08% |
| | Temp. Range | 3559 | 503374 | 55.72 (+/−8.79) | 51.91%/48.09% |
| | Precipitation | 3079 | 350064 | 55.87 (+/−8.62) | 52.09%/47.91% |
| | Snow Depth | 1192 | 164762 | 55.61 (+/−8.64) | 51.74%/48.26% |
| | AQI | 3262 | 448042 | 55.87 (+/−8.82) | 52.40%/47.60% |
| Extreme | Temp. Max | 365 | 7930 | 56.98 (+/−8.49) | 48.54%/51.46% |
| Weather | Temp. Min | 182 | 1962 | 54.56 (+/−8.67) | 56.52%/43.48% |
| | Precipitation | 50 | 263 | 56.80 (+/−8.75) | 44.11%/55.89% |
| | Snowfall | 35 | 187 | 57.97 (+/−7.80) | 60.43%/39.57% |
| | AQI | 172 | 2222 | 55.94 (+/−9.36) | 55.58%/44.42% |
| Temporal | Weekend | 5429 | 940549 | 55.75 (+/−8.76) | 48.89%/51.11% |
| Events | Holidays | 4079 | 99106 | 55.58 (+/−8.78) | 48.84%/51.16% |

TABLE 2
Summary of glucose metrics and how to interpret them.
Glucose metrics are computed on a day of data. Percentiles
computed across the user dataset.

| | Target Range | Percentiles in the dataset across all days | | |
|---|---|---|---|---|
| | for users | 25^{th} | 50^{th} (median) | 75^{th} |
| TIR | >.70 | 0.62 | 0.85 | 0.97 |
| IQR | 13-29 mg/dL | 26.0 | 37.0 | 52.5 |
| MAGE | 41-48 mg/dL | 39.7 | 53.4 | 71.8 |
| % CV | 19-25% | 14.9 | 18.8 | 23.6 |

TABLE 3
Population-level results. Reporting the fixed effect slope from
the corresponding mixed effects model. Bolded when statistically
significant (α < 0.05, with Bonferroni correction).

| | Temp. Max | Weekends | Holidays |
|---|---|---|---|
| TIR | 6.34 × 10⁻⁴ | −1.47 × 10⁻² | −1.90 × 10⁻² |
| | (2.37 × 10⁻⁸) | (7.64 × 10⁻¹²⁴) | (2.23 × 10⁻⁴⁶) |
| IQR | −5.45 × 10⁻² | 9.11 × 10⁻¹ | 7.14 × 10⁻¹ |
| | (2.28 × 10⁻¹²) | (2.37 × 10⁻⁷⁰) | (1.67 × 10⁻⁸) |
| MAGE | −5.95 × 10⁻² | 1.08 × 10⁰ | 4.60 × 10⁻¹ |
| | (8.53 × 10⁻¹) | (6.20 × 10⁻⁶³) | (1.58 × 10⁻³) |
| % CV | −9.27 × 10⁻³ | 2.06 × 10⁻¹ | −4.92 × 10⁻² |
| | (1.67 × 10⁻⁴) | (1.54 × 10⁻⁴⁶) | (1.85 × 10⁻¹) |

TABLE 4
Summary of external events and range of values
that occur in the user dataset. 1^{st} and 99^{th} percentile
values computed from the user dataset.

| | | 1^{st} percentile | 99^{th} percentile |
|---|---|---|---|
| Weather | Temp. Max (° C.) | −6.7 | 38.3 |
| | Temp. Range (° C.) | 0 | 23.3 |
| | Precipitation (mm) | 0 | 46 |
| | Snow Depth (mm) | 0 | 229 |
| | AQI* | 10 | 130 |
| Extreme | Temp. Max | 0 | 1 |
| Weather | Temp. Min | 0 | 1 |
| | Precipitation | 0 | 1 |
| | Snowfall | 0 | 1 |
| | AQI | 0 | 1 |
| Temporal | Weekend | 0 | 1 |
| Events | Holidays | 0 | 1 |

*AQI is on a scale from 0 to 500.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### VII. Examples

In some example embodiments, a computing apparatus comprises memory and one or more processors communicatively coupled to the memory. In some of these example embodiments, the one or more processors are configured to receive sensor data from one or more sensors couplable with a subject body of a subject population comprising a plurality of subject bodies, receive external factor data associated with the subject population, generate, based on applying a first trained machine learning model to the sensor data, the external factor data, and subject body vectors associated with the subject population, a population-level external event impact metric, where the population-level external event impact metric represents a predicted impact of one or more external events on a physiological or other metric of the subject population, generate, based on applying a second trained machine learning model to the population-level external event impact metric and a subject body vector associated with the subject body, a subject-level external event impact metric, wherein the subject-level external event impact metric represents a predicted impact of the one or more external events on the physiological or other metric associated with the subject body, and initiate the performance of one or more prediction-based actions based on the subject-level external event impact metric.

In some of these example embodiments, the sensor data comprises at least one of blood glucose level measurements obtained using a continuous glucose monitoring device or physiological data obtained using a wearable device.

In some of these example embodiments, the external factor data comprises at least one of environmental conditions, weather data, or temporal data.

In some of these example embodiments, the first trained machine learning model is at least one of a linear mixed effects model or a non-linear hierarchical model.

In some of these example embodiments, the second trained machine learning model is at least one of a random forest classifier, a linear model, a deep neural network, an elastic net regression model, a clustering model, a k-means classification model, a support vector machine, or a latent Dirichlet allocation model.

In some of these example embodiments, the first trained machine learning model is trained using historical sensor data and historical external factor data.

In some of these example embodiments, the first trained machine learning model is configured to output one or more of optimized model coefficients or confidence intervals for one or more model coefficients.

In some of these example embodiments, the second trained machine learning model is trained using risk labels assigned to subject body vectors based on segmenting the subject population according to the first trained machine learning model.

In some of these example embodiments, the subject population is segmented according to one or more of random slopes or random intercepts.

In some of these example embodiments, the subject body vector comprises one or more of demographic data, medical history data, or other user data associated with the subject body.

In some of these example embodiments, the one or more prediction-based actions comprise one or more of transmission of alerts to computing devices, transmission of instructions to insulin delivery devices associated with the subject body, adjustments to medical equipment associated with the subject body, or adjustments to allocations of medical, computing, hospital, facility, and/or human resources to the subject body or the subject population.

In some of these example embodiments, the physiological or other metric comprises one or more blood glucose metrics.

In some of these example embodiments, the one or more blood glucose metrics comprise time in range (TIR), interquartile range (IQR), mean amplitude of glycemic excursions (MAGE), percent coefficient of variation (% CV), or number of peaks.

In some of these example embodiments, the subject body is a human.

In some of these example embodiments, the one or more external events are future external events.

In some of these example embodiments, the future external events comprise one or more of a weekend, a holiday, a change in weather conditions, a birthday, or a change in economic conditions.

In some of these example embodiments, the weather conditions comprise precipitation, air quality index (AQI), temperature, snowfall, snow depth, or humidity.

In some of these example embodiments, the subject body vectors associated with the subject population comprise one or more of clinical data or demographic information for subject bodies of the subject population.

In some example embodiments, one or more non-transitory computer-readable storage media include instructions that, when executed by one or more processors, cause the one or more processors to receive sensor data from one or more sensors couplable with a subject body of a subject population comprising a plurality of subject bodies, receive external factor data associated with the subject population, generate, based on applying a first trained machine learning model to the sensor data, the external factor data, and subject body vectors associated with the subject population, a population-level external event impact metric, wherein the population-level external event impact metric represents a predicted impact of one or more external events on a physiological or other metric of the subject population, generate, based on applying a second trained machine learning model to the population-level external event impact metric and a subject body vector associated with the subject body, a subject-level external event impact metric, wherein the subject-level external event impact metric represents a predicted impact of the one or more external events on the physiological or other metric associated with the subject body, and initiate the performance of one or more prediction-based actions based on the subject-level external event impact metric.

In some example embodiments, a computer-implemented method comprises receiving, by one or more processors, sensor data from one or more sensors couplable with a subject body of a subject population comprising a plurality of subject bodies, receiving, by the one or more processors, external factor data associated with the subject population, generating, by the one or more processors and based on applying a first trained machine learning model to the sensor data, the external factor data, and subject body vectors associated with the subject population, a population-level external event impact metric, wherein the population-level external event impact metric represents a predicted impact of one or more external events on a physiological or other metric of the subject population, generating, by the one or more processors and based on applying a second trained machine learning model to the population-level external event impact metric and a subject body vector associated with the subject body, a subject-level external event impact metric, wherein the subject-level external event impact metric represents a predicted impact of the one or more external events on the physiological or other metric associated with the subject body, and initiating, by the one or more processors, the performance of one or more prediction-based actions based on the subject-level external event impact metric.

1. A computing apparatus comprising memory and one or more processors communicatively coupled to the memory, the one or more processors configured to:
receive sensor data from one or more sensors couplable with a subject body of a subject population comprising a plurality of subject bodies;
receive external factor data associated with the subject population;
generate, based on applying a first trained machine learning model to the sensor data, the external factor data, and subject body vectors associated with the subject population, a population-level external event impact metric, wherein the population-level external event impact metric represents a predicted impact of one or more external events on a physiological or other metric of the subject population;
generate, based on applying a second trained machine learning model to the population-level external event impact metric and a subject body vector associated with the subject body, a subject-level external event impact metric, wherein the subject-level external event impact metric represents a predicted impact of the one or more external events on the physiological or other metric associated with the subject body; and
initiate the performance of one or more prediction-based actions based on the subject-level external event impact metric.

2. The computing apparatus of claim 1, wherein the sensor data comprises at least one of blood glucose level measurements obtained using a continuous glucose monitoring device or physiological data obtained using a wearable device.

3. The computing apparatus of claim 1, wherein the external factor data comprises at least one of environmental conditions, weather data, or temporal data.

4. The computing apparatus of claim 1, wherein the first trained machine learning model is at least one of a linear mixed effects model or a non-linear hierarchical model.

5. The computing apparatus of claim 1, wherein the second trained machine learning model is at least one of a random forest classifier, a linear model, a deep neural network, an elastic net regression model, a clustering model, a k-means classification model, a support vector machine, or a latent Dirichlet allocation model.

6. The computing apparatus of claim 1, wherein the first trained machine learning model is trained using historical sensor data and historical external factor data.

7. The computing apparatus of claim 6, wherein the first trained machine learning model is configured to output one or more of optimized model coefficients or confidence intervals for one or more model coefficients.

8. The computing apparatus of claim 1, wherein the second trained machine learning model is trained using risk labels assigned to subject body vectors based on segmenting the subject population according to the first trained machine learning model.

9. The computing apparatus of claim 8, wherein the subject population is segmented according to one or more of random slopes or random intercepts.

10. The computing apparatus of claim 1, wherein the subject body vector comprises one or more of demographic data, medical history data, or other user data associated with the subject body.

11. The computing apparatus of claim 1, wherein the one or more prediction-based actions comprise one or more of transmission of alerts to computing devices, transmission of instructions to insulin delivery devices associated with the subject body, adjustments to medical equipment associated with the subject body, or adjustments to allocations of medical, computing, hospital, facility, and/or human resources to the subject body or the subject population.

12. The computing apparatus of claim 1, wherein the physiological or other metric comprises one or more blood glucose metrics.

13. The computing apparatus of claim 12, wherein the one or more blood glucose metrics comprise time in range (TIR), interquartile range (IQR), mean amplitude of glycemic excursions (MAGE), percent coefficient of variation (% CV), or number of peaks.

14. The computing apparatus of claim 1, wherein the subject body is a human.

15. The computing apparatus of claim 1, wherein the one or more external events are future external events.

16. The computing apparatus of claim 15, wherein the future external events comprise one or more of a weekend, a holiday, a change in weather conditions, a birthday, or a change in economic conditions.

17. The computing apparatus of claim 16, wherein the weather conditions comprise precipitation, air quality index (AQI), temperature, snowfall, snow depth, or humidity.

18. The computing apparatus of claim 1, wherein the subject body vectors associated with the subject population comprise one or more of clinical data or demographic information for subject bodies of the subject population.

19. One or more non-transitory computer-readable storage media including instructions that, when executed by one or more processors, cause the one or more processors to:
receive sensor data from one or more sensors couplable with a subject body of a subject population comprising a plurality of subject bodies;
receive external factor data associated with the subject population;
generate, based on applying a first trained machine learning model to the sensor data, the external factor data, and subject body vectors associated with the subject population, a population-level external event impact metric, wherein the population-level external event impact metric represents a predicted impact of one or more external events on a physiological or other metric of the subject population;
generate, based on applying a second trained machine learning model to the population-level external event impact metric and a subject body vector associated with the subject body, a subject-level external event impact metric, wherein the subject-level external event impact metric represents a predicted impact of the one or more external events on the physiological or other metric associated with the subject body; and
initiate the performance of one or more prediction-based actions based on the subject-level external event impact metric.

20. A computer-implemented method comprising:
receiving, by one or more processors, sensor data from one or more sensors couplable with a subject body of a subject population comprising a plurality of subject bodies;
receiving, by the one or more processors, external factor data associated with the subject population;
generating, by the one or more processors and based on applying a first trained machine learning model to the sensor data, the external factor data, and subject body vectors associated with the subject population, a population-level external event impact metric, wherein the population-level external event impact metric represents a predicted impact of one or more external events on a physiological or other metric of the subject population;
generating, by the one or more processors and based on applying a second trained machine learning model to the population-level external event impact metric and a subject body vector associated with the subject body, a subject-level external event impact metric, wherein the subject-level external event impact metric represents a predicted impact of the one or more external events on the physiological or other metric associated with the subject body; and
initiating, by the one or more processors, the performance of one or more prediction-based actions based on the subject-level external event impact metric.
